(19) United States
(12) Patent Application Publication
(10) Pub. No.: US 2021/0210213 A1
(43) Pub. Date: Jul. 8, 2021

(54) METHOD, APPARATUS, MEDIUM AND ELECTRONIC DEVICE FOR RECOMMENDING MEDICINE

(71) Applicant: BOE TECHNOLOGY GROUP CO., LTD., Beijing (CN)

(72) Inventor: Yafei DAI, Beijing (CN)

(21) Appl. No.: 16/768,326

(22) PCT Filed: Jan. 15, 2020
(86) PCT No.: PCT/CN2020/072293

§ 371 Date: May 29, 2020

(30) Foreign Application Priority Data
Jan. 15, 2019 (CN) 201910036667.X

(51) Int. Cl. G16H 50/70; G16H 10/60; G16H 50/20; G16H 20/10; G16H 70/40; G16H 15/00; G06F 16/9535; G06F 40/289

(52) U.S. Cl. CPC G16H 50/70; G16H 10/60; G16H 50/20; G06F 40/289; G16H 70/40; G16H 15/00; G06F 16/9535; G16H 20/10

(57) ABSTRACT

The present disclosure relates to the field of big data technology and, in particular, to a method and an apparatus for recommending medicine, a non-transitory computer-readable storage medium, and an electronic device that implement a method that includes: determining at least one consultation keyword based on obtained consultation information on a patient's medication; inputting the at least one consultation keyword into a medicine knowledge graph, performing medicine filtering through querying related information in the medicine knowledge graph; and outputting a filter-out target recommended medicine to complete the medicine recommendation.

### CROSS REFERENCE TO RELATED APPLICATIONS

This application is a national phase application under 35 U.S.C. § 371 of International Patent Application No. PCT/CN2020/072293, filed on Jan. 15, 2020, which claims the benefit of and priority to Chinese Patent Application No. 201910036667.X filed on Jan. 15, 2019, the contents of both of which being incorporated by reference in their entireties herein.

### TECHNICAL FIELD

The present disclosure relates to the field of knowledge graph technology and, in particular, to a method for recommending medicine, an apparatus for recommending medicine, as well as a non-transitory computer-readable storage medium and an electronic device that implement the method for recommending medicine.

### BACKGROUND

Conventional methods for purchasing medicine usually include purchasing in hospitals or physical pharmacies, or through online pharmacies.

When purchasing medicines in hospitals/physical pharmacies, doctors/shopping staff recommend medicines for patients based on medical experience. In this method for recommending medicine, the accuracy of medicine recommendation needs to be improved.

When purchasing medicines through an online pharmacy, the purchaser makes inquiries in the online pharmacy for a certain disease. For instance, a search can be made through major medicine websites on the Internet, and the results may include brands, imported or not, externally used or not, and the like. In addition, the results can be sorted by sales volume or price, so that the purchasers can choose their own medicines.

It should be noted that the information disclosed in the above background section is only for enhancing the understanding of the background of the present disclosure, and therefore may include information that does not constitute prior art known to those of ordinary skill in the art.

### SUMMARY

The present disclosure is directed to provide a method for recommending medicine, an apparatus for recommending medicine, as well as a non-transitory computer-readable storage medium, and an electronic device that implement the method for recommending medicine.

Other features and advantages of the present disclosure will become apparent through the following detailed description, or partly learned through practice of the present disclosure.

According to a first aspect of embodiments of the disclosure, there is provided a method for recommending medicine. The method includes:

acquiring consultation information on medication of a patient;

querying a preset medicine knowledge graph based on the consultation information to filter out a target recommended medicine and unrecommended medicine information; and

outputting the target recommended medicine and the unrecommended medicine information.

According to an exemplary embodiment of the disclosure, based on the forgoing embodiments, the unrecommended medicine information includes a name of a unrecommended medicine and a reason for non-recommendation.

According to an exemplary embodiment of the disclosure, based on the forgoing embodiments, the querying the preset medicine knowledge graph based on the consultation information includes:

determining at least one consulting keyword based on the consulting information; and

inputting the at least one consulting keyword into the preset medicine knowledge graph, and performing medicine filtering through related information search in the medicine knowledge graph.

According to an exemplary embodiment of the disclosure, based on the forgoing embodiments, a mapping table between text and symbols is imported into the medicine knowledge graph, and querying the preset medicine knowledge graph based on the consultation information further includes:

converting the consulting keyword into a symbol based on the mapping table; and

inputting the symbol into the medicine knowledge graph to perform direct match, and returning a result of the medicine filtering.

According to an exemplary embodiment of the disclosure, based on the forgoing embodiments, the determining at least one consultation keyword based on the consultation information includes:

obtaining, from the consultation information on medication of the patient by means of natural language processing, at least one of following consultation keywords: a keyword related to the patient's disease, a keyword related to the patient's characteristics, a keyword related to the patient's appeal, or a keyword related to the patient's response to medicines.

According to an exemplary embodiment of the disclosure, based on the forgoing embodiments, the performing medicine filtering through related information search in the medicine knowledge graph includes:

determining, according to the keyword related to the patient's disease, a corresponding disease name entity as a target disease in the medicine knowledge graph;

obtaining, based on the medicine knowledge graph, all medicine name entities associated with the target disease as first recommended medicines, and obtaining all medicine usage precaution entities associated with the first be recommended medicines; and

determining the target recommended medicine applicable to the patient, a unrecommended medicine not applicable to the patient and a reason for non-recommendation by filtering the first recommended medicines according to at least one of the keyword related to the patient's characteristics, the keyword related to the patient's appeal, or the keyword related to the patient's response to medicines and the obtained medicine usage precaution entities.

According to an exemplary embodiment of the disclosure, based on the forgoing embodiments, the filtering the first recommended medicines includes:

determining second recommended medicines applicable to the patient by performing a first filtering on the first recommended medicines according to the keyword related to the patient's characteristics and the medicine usage precaution entities;

determining the target recommended medicine applicable to the patient by performing a second filtering on the second recommended medicines according to the keyword related to the patient's response to medicines and the medicine usage precaution entities; and

determining the unrecommended medicine and the reason for non-recommendation based on results of the first filtering and the second filtering.

According to an exemplary embodiment of the disclosure, based on the forgoing embodiments, the keyword related to the patient's disease includes a name of a disease corresponding to the patient, the keyword related to the patient's characteristics includes age information and/or whether the patient is pregnant or not, the keyword related to the patient's appeal includes medicines that the patient does not want to use and/or characteristics of medicines required by the patient; and the keyword related to the patient's response to medicines includes allergy history information.

According to an exemplary embodiment of the disclosure, based on the forgoing embodiments, the outputting the target recommended medicine includes:

outputting a name of the target recommended medicine by playing voice message, and/or, displaying the name of the target recommended medicine by text.

According to an exemplary embodiment of the disclosure, based on the forgoing embodiments, before querying a preset medicine knowledge graph based on the consultation information, the method further includes:

collecting medicine data, the medicine data including names of medicines, names of diseases to which the medicines apply, and precautions for usage of the medicines; and

generating the preset medicine knowledge graph by determining a data triplet of “entity-relationship-entity” in which the names of diseases in the medicine data serve as a starting point, and the names of medicines corresponding to the starting point and the precautions for usage of the medicines are associated with the starting point.

According to an exemplary embodiment of the disclosure, based on the forgoing embodiments, the entities in the data triplet include: a disease name entity, a medicine name entity, or a medicine usage precaution entity.

According to an exemplary embodiment of the disclosure, based on the forgoing embodiments, the relationship in the data triplet includes: a treatment relationship between the disease name entity and the medicine name entity, or a usage description relationship between the medicine name entity and the medicine usage precaution entity.

According to an exemplary embodiment of the disclosure, based on the forgoing embodiments, the medicine data further includes a medicine dosage, and the method further includes outputting the medicine dosage along with the target recommended medicine to a user.

According to a second aspect of embodiments of the disclosure, there is provided an apparatus for recommending medicine. The apparatus includes:

an inputting module configured to acquire consultation information on medication of a patient;

a querying module configured to query a preset medicine knowledge graph based on the consultation information to filter out a target recommended medicine and unrecommended medicine information; and

an outputting module configured to output the target recommended medicine and the unrecommended medicine information.

According to an exemplary embodiment of the disclosure, based on the forgoing embodiments, the unrecommended medicine information includes a name of a unrecommended medicine and a reason for non-recommendation.

According to an exemplary embodiment of the disclosure, based on the forgoing embodiments, the querying module is further configured to: determine at least one consulting keyword based on the consulting information; input the at least one consulting keyword into the preset medicine knowledge graph, and perform medicine filtering through related information search in the medicine knowledge graph.

According to an exemplary embodiment of the disclosure, based on the forgoing embodiments, a mapping table between text and symbols is imported into the medicine knowledge graph, and the querying module is further configured to: convert the consulting keyword into a symbol based on the mapping table; input the symbol into the medicine knowledge graph to perform direct match, and return a result of the medicine filtering.

According to an exemplary embodiment of the disclosure, based on the forgoing embodiments, the apparatus further includes:

a constructing module configured to collect medicine data including names of medicines, names of diseases to which the medicines apply, and precautions for usage of the medicines; and generate the preset medicine knowledge graph by determining a data triplet of “entity-relationship-entity” in which the names of diseases in the medicine data serve as a starting point, and the names of medicines corresponding to the starting point and the precautions for usage of the medicines are associated with the starting point.

According to a third aspect of embodiments of the disclosure, there is provided an electronic device, including:

a processor; and

a memory storing a computer program,

the processor, when executing the computer program, is configured to implement the method according to any embodiment of the first aspect.

According to a fourth aspect of embodiments of the disclosure, there is provided a computer storage medium storing a computer program, wherein the computer program, when being executed by a processor, causes the processor to implement the method according to any embodiment of the first aspect.

It should be understood that the above general description and the following detailed description are only exemplary and explanatory, and do not limit the present disclosure.

This section provides an overview of various implementations or examples of the technology described in this disclosure, and is not a comprehensive disclosure of the full scope or all features of the disclosed technology.

### BRIEF DESCRIPTION OF THE DRAWINGS

The drawings herein are incorporated into and constitute a part of this specification, illustrate embodiments consistent with this disclosure, and are used together with the specification to explain the principles of this disclosure. Understandably, the drawings in the following description are only some embodiments of the present disclosure. For those of ordinary skill in the art, without paying any creative work, other drawings can be obtained based on these drawings.

FIG. 1 schematically illustrates a flowchart of a method for recommending medicine according to an exemplary embodiment of the present disclosure;

FIG. 2 schematically illustrates a flowchart of a method for recommending medicine according to another exemplary embodiment of the present disclosure;

FIG. 3 schematically illustrates a flowchart of a method for constructing a medicine knowledge graph according to an exemplary embodiment of the present disclosure;

FIG. 4 schematically illustrates a medicine knowledge graph constructed based on Neo4j according to an exemplary embodiment of the present disclosure;

FIG. 5 schematically illustrates a mapping table imported in the medicine knowledge graph according to an exemplary embodiment of the present disclosure;

FIG. 6 schematically illustrates the data storage form of the medicine knowledge graph according to an exemplary embodiment of the present disclosure;

FIG. 7 schematically illustrates a flowchart of a method for querying related information through the medicine knowledge graph according to an exemplary embodiment of the present disclosure;

FIG. 8 schematically illustrates a medicine recommendation result output in text according to an exemplary embodiment of the present disclosure;

FIG. 9 schematically illustrates a block diagram of an apparatus for recommending medicine according to an exemplary embodiment of the present disclosure;

FIG. 10 schematically illustrates a computer-readable storage medium for implementing the above method for constructing the medicine knowledge graph;

FIG. 11 schematically illustrates a block diagram of an electronic device for implementing the above method for constructing the medicine knowledge graph.

### DETAILED DESCRIPTION

Exemplary embodiments will now be described more fully with reference to the drawings. However, the exemplary embodiments can be implemented in various forms, and should not be construed as being limited to the examples set forth herein. On the contrary, these embodiments are provided to make the disclosure more comprehensive and complete, and fully conveys the concept of the exemplary embodiments for those skilled in the art. The described features, structures, or characteristics may be combined in one or more embodiments in any suitable manner.

In addition, the drawings are only schematic illustrations of the present disclosure and are not necessarily drawn to scale. The same reference numerals in the drawings denote the same or similar parts, and thus their repeated description will be omitted. Some of the block diagrams shown in the drawings are functional entities and do not necessarily have to correspond to physically or logically independent entities. These functional entities may be implemented in the form of software, or implemented in one or more hardware modules or integrated circuits, or implemented in different networks and/or processor devices and/or microcontroller devices.

The block diagrams shown in the drawings are merely functional entities and do not necessarily have to correspond to physically independent entities. That is, these functional entities can be implemented in the form of software, or implemented in one or more hardware modules or integrated circuits, or implemented in different networks and/or processor devices and/or microcontroller devices entity.

The flow charts shown in the drawings are only exemplary illustrations, and it is not necessary to include all contents and operations/steps, nor to be executed in the order as described. For example, some operations/steps can also be decomposed, and some operations/steps can be merged or partially merged, so the order of actual execution may change according to the actual situation.

It should be noted that although the steps of the method in the present disclosure are described in a specific order in the drawings, this does not require or imply that the steps must be performed in the specific order, or all the steps shown must be performed to achieve the desired result. Additionally or alternatively, some steps may be omitted, multiple steps may be combined into one step for execution, and/or one step may be decomposed into multiple steps for execution, and the like. In addition, it is also easy to understand that these steps may be performed, for example, synchronously or asynchronously in multiple modules/processes/threads.

In the conventional method for recommending medicine through online pharmacies, due to the complexity of the medicine information existed online, it is not possible to efficiently recommend the corresponding medicine for the target diseases of medicine buyers. For example, as to a brand “a” in an online pharmacy, there are 494 kinds of cold medicines belonging to the brand “a”. Further, specific medicine information needs to be checked in turn, and the huge amount of data makes the medicine query lose the advantage of convenient and fast online shopping. This is only a medicine for one disease. In reality, many diseases exist at the same time. For example, a patient may have both gastroenteritis and a cold. In such case, the patient may need to inquire about two or more diseases. However, many medicines cannot be taken at the same time. Moreover, when a patient is allergic to, for example, penicillin, such situation also requires manual inspection through detailed reading of the instructions.

This exemplary embodiment firstly provides a method for recommending medicine. FIG. 1 schematically illustrates a flowchart of a method for recommending medicine according to an exemplary embodiment of the present disclosure, at least to a certain extent to overcome the above problem caused by limitations and defects of related art. In an embodiment, the execution subject of the method for recommending medicine provided in this embodiment may be a device with a calculation processing function, such as a server.

Referring to FIG. 1, the method may include the following steps.

In step S101, consultation information on medication of a patient is acquired.

In some embodiments, the consultation information here may be input when the patient is in use, or may be directly called from a memory.

In step S102, a preset medicine knowledge graph is queried based on the consultation information to filter out a target recommended medicine and unrecommended medicine information.

In some embodiments, the medicine knowledge graph here is constructed based on medical literature knowledge and clinical practical data. For example, the medicine knowledge graph is pre-built and stored, and can be directly accessed and queried when used.

In step S103, the target recommended medicine and the unrecommended medicine information are output.

In some embodiments, the unrecommended medicine information includes the name of the unrecommended medicine.

In related art, only applicable medicines are recommended, and it is not clear whether any medicines are not included in the recommendation, or not included in the knowledge graph, or not suitable for use by patients. According to the embodiments of the present application, not only the applicable medicines are recommended, but information about unrecommended medicines is given, including, for example, the name of the medicine or the name thereof and the reason for the non-recommendation, so as to make it easier for users to understand the specific situation, thereby improving the user experience.

In some embodiments, the unrecommended medicine information includes the name of the unrecommended medicine and the reason for the non-recommendation.

In the technical solution provided by the embodiment shown in FIG. 1, the medicine knowledge graph is queried based on the obtained consultation information about the patient's medication to filter out the target recommended medicine and unrecommended medicine information for output, which can improve the accuracy and efficiency of medicine recommendation, while improving the user experience.

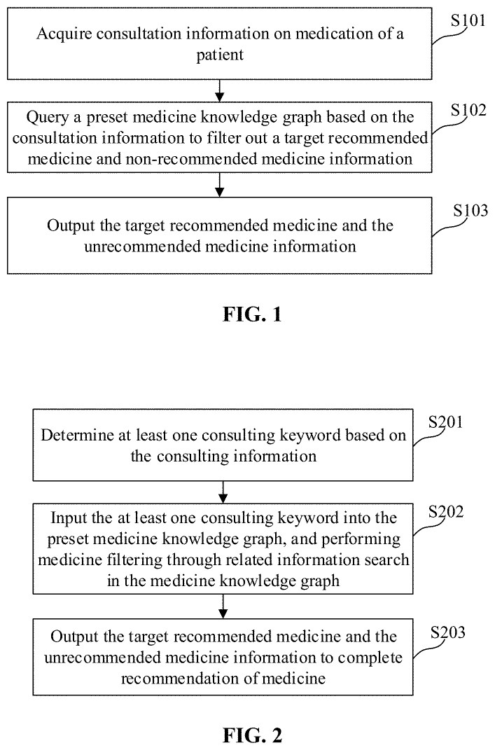

FIG. 2 schematically illustrates a flowchart of a method for recommending medicine according to another exemplary embodiment of the present disclosure. In an embodiment, the execution subject of the method for recommending medicine provided in this embodiment may be a device with a calculation processing function, such as a server.

Referring to FIG. 2, the method may include the following steps.

In step S201, at least one consultation keyword is determined according to the acquired consultation information about the patient's medication.

In step S202, the at least one consulting keyword is input into the medicine knowledge graph, and medicine filtering is performed through related information search in the medicine knowledge graph.

In step S203, the target recommended medicine and unrecommended medicine information to complete the recommendation of the medicine.

According to the technical solution provided in the embodiment shown in FIG. 2, at least one consultation keyword is determined based on the acquired consultation information about the patient's medication and, then, input to the medicine knowledge graph to perform medicine filtering through related information search in the medicine knowledge graph. On one hand, medicine recommendation is implemented through the medicine knowledge graph constructed based on medical literature knowledge and clinical practical data to, thereby improving the accuracy of medicine recommendation. On the other hand, the associated query is performed in the medicine knowledge graph through at least one consultation keyword, the target recommended medicine corresponding to the above-mentioned consultation information can be efficiently obtained, thereby helping to improve the efficiency of medicine recommendation. On the other hand, while recommending applicable medicines, information about medicines that are not recommended is also output, including the name of the medicine and the reason for not recommending it, so as to make it easier for users to understand the specific situation and enhance the user experience.

The specific implementation of each step of the embodiment shown in FIG. 2 is described in more detail below.

In an exemplary embodiment of the present disclosure, the usage scenario of the embodiment shown in FIG. 2 may be that the patient or family of the patient (which may be collectively referred to as “user”) proposes information about the patient's medication through keyboard input or voice input. Then, in step S201, the consultation information about the patient's medication is acquired, and the consultation keywords are obtained from the consultation information by means of natural language processing (NLP). Exemplarily, the obtained keywords include, but are not limited to, a keyword related to the patient's disease, a keyword related to the patient's characteristics, a keyword related to the patient's appeal, or a keyword related to the patient's response to medicines. Exemplarily, the consultation keywords belonging to the same user are taken as a group, so as to input a group of consultation keywords corresponding to the same user into the medicine knowledge graph, and then the medicine filtering corresponding to the use is completed through related information search in the medicine knowledge graph.

In an exemplary embodiment, among the consultation keywords obtained from the consultation information about the patient's medication through NLP, the keyword related to the patient's disease may be a disease name corresponding to the patient; the keyword related to the patient's characteristics may include: age information, whether the patient is pregnant or not; the keyword related to the patient's appeal may include: medicines that the patient does not want to use (for example, do not want to use medicines produced by an enterprise), the characteristics of the medicines required by the patient (for example, it is desired that the radiation absorption dose of the medicine is relatively large), and the like; the keyword related to the patient's response to medicines may include allergy history information and the like.

Exemplarily, according to the consultation information about the patient's medication entered by the user, for example, “I am 72 years old, have a cold recently, and are allergic to penicillin, please recommend the medicines I can use.” The above consultation information may be segmented through NLP, and part-of-speech tagging and other operations may be carried out. The keywords obtained through the consultation may include cold (disease keyword), elderly (patient characteristic keyword), penicillin allergy (the keyword related to the patient's response to medicines), and the like.

In related art, the patient's answers are often obtained through multiple rounds of question and answer, for example, whether she is pregnant or whether he/she is determined to have the disease. In this embodiment, because the question framework has been determined, the procedure is easier to handle and get answers. However, these questions are difficult to fully cover the filtering information that the patient wants to express. Taking the text “I was recently in pregnancy” as example, although the patient is not pregnant, it is still necessary to avoid the intake of some medicines. The above-mentioned way through NLP can deal with such problems, more in line with the patient's way of thinking and expression, so that the medicines filtering can be more accurate. In some embodiments of the present disclosure, the text is first identified with a named entity and the relationship is extracted, the keyword “pregnancy period” is determined, the relationship is “yes”, and then the entity link technology is used to link the entity to the medicine information in the knowledge base. When corresponding label is “prohibited”, “prudent” medication, the medicine may be classified as not recommended.

After obtaining the consultation keywords, the consultation keywords are input into the medicine knowledge graph in step S202.

In an exemplary embodiment, before performing step S202, the technical solution provided by this embodiment further includes constructing a medicine knowledge graph.

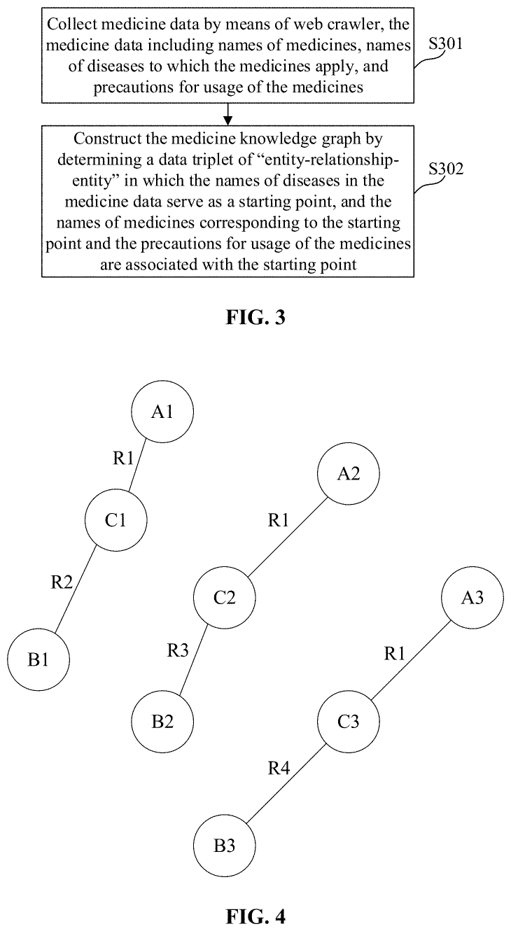

Exemplarily, FIG. 3 schematically illustrates a flowchart of a method for constructing a medicine knowledge graph according to an exemplary embodiment of the present disclosure.

Referring to FIG. 3, the method includes steps S301-S302.

In step S301, medicine data is collected and includes names of medicines, names of diseases to which the medicines apply, and precautions for usage of the medicines.

For example, a large number of medicine data may be collected through web crawlers.

In an exemplary embodiment, medicine-related data is crawled from major medicine and disease websites, and the acquired medicine data includes names of medicines, names of diseases to which the medicines apply, and precautions for usage of the medicines. Exemplarily, for a medicine “a”, the medicine name “A” thereof, the disease name “b” of the disease to which the medicine a applies, and also the precautions for usage of the medicine are collected. For example, the precautions may include that “the medicine should be used by pregnant women with caution”, “the medicine cannot be used along with the medicine with the name M”, and the like.

In an exemplary embodiment, the above medicine knowledge graph is constructed based on medical literature knowledge and clinical practical data. In addition, medical uniform names may be used for the above-mentioned medicine names, disease names and the like, so as to avoid the problem of using different medicine names for the same medicine, thereby helping to improve the accuracy of medicine recommendation.

In an exemplary embodiment, the acquired medicine data, in addition to the name of medicine, the name of disease to which the medicine applies, and precautions for usage of medicine, may also include the dosage of medicine. In this way, when the medicine is recommended to the user through the medicine knowledge graph, the information such as the dosage of medicines can be simultaneously pushed to the user, thereby improving the convenience of the user in recommending medicines through the medicine knowledge graph.

In an exemplary embodiment, the crawled data may be cleaned to avoid information redundancy, which may in turn lead to information redundancy of the constructed medicine knowledge graph, thereby helping to improve the efficiency and accuracy of medicine recommendation using the medicine knowledge graph.

In step S302, the disease name in the medicine data is used as a starting point, the medicine name corresponding to the starting point and the precautions for usage of the medicine are associated to determine a data triple of “entity-relationship-entity”, so as to build a medical knowledge graph.

In an exemplary embodiment of the medicine knowledge graph, the “entities” in the data triplet may include disease name entities, medicine name entities, or medicine usage precaution entities. Correspondingly, the “relationship” in the above data triplet may include: a treatment relationship between the disease name entity and the medicine name entity, a usage description relationship between the medicine name entity and the medicine usage precaution entity, and the like. The medicine knowledge graph constructed by using data triplets has the logical structure ability of knowledge reasoning. Compared with the medicine search process based on the search engine in the prior art, the medicine knowledge graph constructed by the data triplet for medicine search can better understand the semantic scope domain, thereby improving the accuracy of medicine search.

In an exemplary embodiment, the present application organizes the acquired medical data into a graph logic data structure based on the concept of graph theory. Graph theory is a branch of mathematics, with graph as the research object. The graph in graph theory is a figure composed of a number of given points and a line connecting two points. This figure is usually used to describe a certain relationship between certain objects, with points representing objects and the line which connects two points indicating that there is relationship between the two corresponding objects.

Based on the concept of graph theory, nodes may include entities such as disease name, medicine name, medicine usage and dosage, and relationships may include medicines, usage and dosage, specifications and the like. In this way, medical data may be thus represented and stored through these nodes and relationships. The data triplet “entity-relationship-entity” is determined, and then the acquired medical data can be connected together to obtain a relationship network, thereby constructing the medicine knowledge graph. For example, taking Gankan™, a medicine for treating upper respiratory tract infections, as an example, the information contained in the medicine specification can be stored in the medicine knowledge graph through the above-mentioned “entity-relationship-entity” data triplet: “upper respiratory tract infection” (entity)—“medicine” (relationship)—“Gankang” (entity), “Gankang” (entity)—“contraindications for use” (relationship)—“prohibited for those having severe liver and kidney dysfunction” (entity), “Gankang” (entity)—“side effects” (relationship)—“occasionally rash, urticaria, medicine fever and neutropenia” (entity).

In an exemplary embodiment, a graph database storage system tool (such as Neo4j) may be used to implement construction of the medicine knowledge graph. Neo4j is a high-performance NOSQL database (Not Only SQL, generally referred to as a non-relational database), which stores structured data on the network instead of tables. Neo4j can also be seen as a high-performance graph engine. Based on neo4j, some commonly used graph algorithms can be adopted, such as the link prediction algorithm which is used for determining the intimacy between two adjacent nodes.

FIG. 4 schematically illustrates a medicine knowledge graph constructed based on Neo4j according to an exemplary embodiment of the present disclosure. As shown in FIG. 4, the medicine knowledge graph is composed of vertices and edges, and each vertex corresponds to the “entity” in the above data triplet, including, for example, the disease name entities A1-A3, the medicine usage precaution entities B1-B3, and medicine name entities C1-C3; each edge corresponds to the “relationship” in the above data triplet, including for example, treatment relationship R1 between the medicine name entities A1-A3 and the medicine name entities C1-C3, the usage description relationships R2-R4 between the medicine name entities C1-C3 and the medicine usage precaution entities B1-B3.

In an exemplary embodiment, a mapping table between text and symbols is imported into the medicine knowledge graph. When the medicine knowledge graph is constructed, texts such as disease name entity, medicine name entity and medicine usage precaution entity are converted into symbols for storage. Correspondingly, when querying the medicine knowledge graph based on the consulting information, the consulting keywords are also converted into symbols based on the above mapping table, and the symbols are input into the medicine knowledge graph for direct matching, so as to return the result of medicine filtering.

In a specific example, the medicine knowledge graph constructed in the above manner includes a total of more than 520,000 medicines, more than 1.14 million entity nodes, and a total number of three triples (that is, the total number of knowledge items that express the relationship between nodes) is more than 12.15 million. A total number of medicine labels is 78, including information such as product ingredients, usage and dosage, indications, contraindications, pharmacological effects, interactions and the like. It takes less than a minute to import all those knowledge items into neo4j for storage.

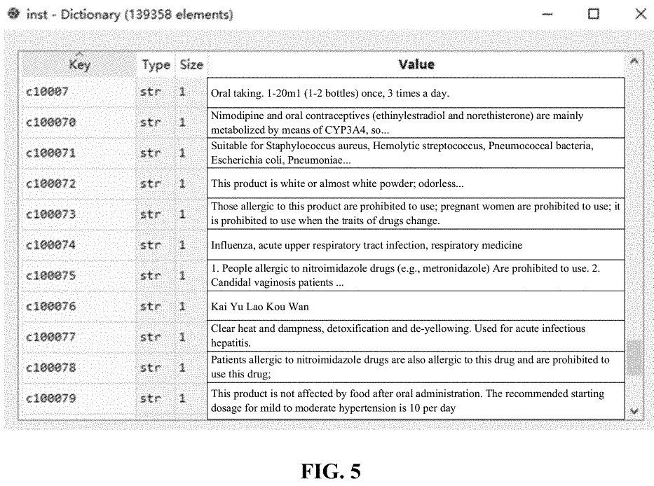

FIG. 5 schematically illustrates a mapping table imported in the medicine knowledge graph according to an exemplary embodiment of the present disclosure. Referring to FIG. 5, the leftmost column “Key” represents the symbol stored in the medicine knowledge graph, and the rightmost column “Value” represents text. In addition, “Type” represents the property of “Value”, for example, “str” represents a character string. “Size” represents the size of “Value”, for example, 1 represents that “Value” includes 1 character string. Taking the last third row in the table shown in FIG. 5 as an example, the usage precaution entity obtained by collecting medicine instructions include “Clear heat and dampness, detoxification and de-yellowing. Used for acute infectious hepatitis”. Its corresponding symbol is obtained, for example, by sequential numbering, as c100077, and is imported into neo4j for storage.

FIG. 6 schematically illustrates the data storage form of the medicine knowledge graph according to an exemplary embodiment of the present disclosure. Referring to FIG. 6, columns A and C respectively represent entities in the above data triplet, for example, corresponding to disease name entities and the medicine name entity; columns B and D are the name and type of the relationship, for example, both are r2, which indicates the treatment relationship between the columns A and C. With reference to FIG. 5, the symbols in columns A and C can be determined by collecting the medicine specification and for example by serial numbering.

In the technical solutions provided by the embodiments shown in FIGS. 4-6, by importing a text-to-symbol mapping table into the medicine knowledge graph constructed by Neo4j, the retrieval speed of related information search can be improved, and the retrieval results can be returned through direct matching in the imported mapping table. Correspondingly, by using the schema index in neo4j, the time for the method of recommending medicine according to the disclosed embodiments to return the result can reach milliseconds, thereby further improving the efficiency and response speed of medicine recommendation and, thus, improving user experience.

With continued reference to FIG. 2, in step S202, the at least one consultation keyword is input to the medicine knowledge graph, and medicine filtering is performed through related information search in the medicine knowledge graph.

In an exemplary embodiment, a set of consultation keywords (at least one consultation keyword) corresponding to the same consultation information is input into the above medicine knowledge graph constructed in this embodiment, using the above medicine knowledge graph, the above set of consultation keywords is analyzed from the perspective of “relationship”, so as to realize the purpose of related information search through the medicine knowledge graph, thereby completing the medicine filtering to obtain the target recommended medicines corresponding to the above set of consultation keywords.

Exemplarily, in the specific implementation of step S201, according to the consultation information about the patient's medication entered by the user, for example, “I am 72 years old, have a cold recently, and are allergic to penicillin, please recommend the medicines I can use.” The above consultation information may be segmented through NLP, and part-of-speech tagging and other operations may be carried out. The keywords obtained through the consultation may include cold (disease keyword), elderly (patient characteristic keyword), penicillin allergy (the keyword related to the patient's response to medicines), and the like.

Then, in this embodiment, the above-mentioned medicine knowledge graph is connected based on the Python platform to compile and package the query filtering program. The above medicine knowledge graph can be connected in Python for query, so as to filter out inappropriate medicines (for example, the entity “elderly patient prohibited” in the relationship of “elderly medicine”, and the entity including “penicillin” in the relationship of “medicine description” or “contraindications”) are listed under the unrecommended label. That is to say, through entity recognition, relationship extraction and recognition reasoning, the final query result can be obtained.

Compared with single search based on one or several keywords in the medicine filtering process using a conventional search engine, according to the technical solution provided in this embodiment, the knowledge graph is used to perform related information search based on a set of consultation keywords. Accordingly, based on this technical solution, complex related information can be retrieved more accurately, thereby improving the quality of medicine search, and thus helping to improve the accuracy of medicine recommendation. At the same time, the use of medicine knowledge graph for related information search is conducive to improving the efficiency of medicine recommendation.

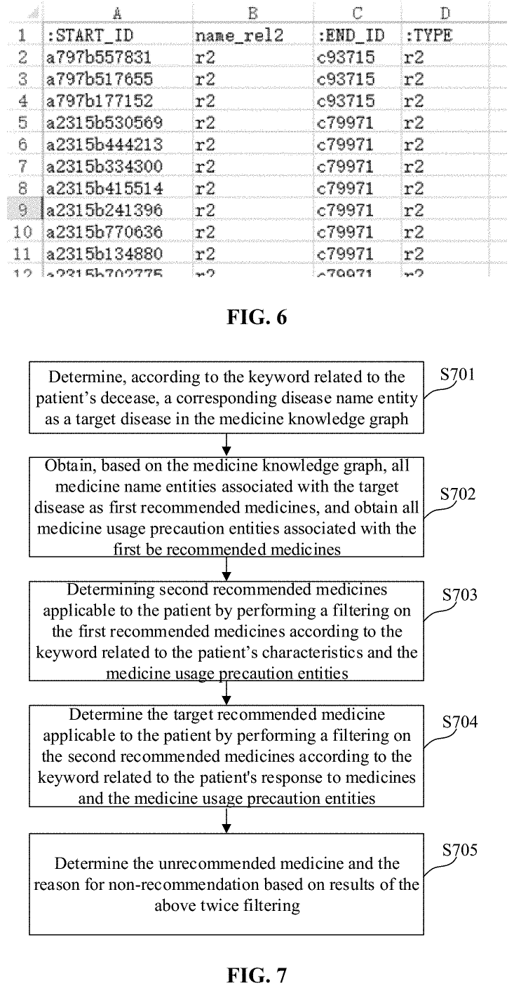

In an exemplary embodiment, FIG. 7 schematically illustrates a flowchart of a method for querying related information through the medicine knowledge graph according to an exemplary embodiment of the present disclosure. The specific implementation of step S202 will be described below with reference to FIG. 7.

Referring to FIG. 7, the method includes steps S701-S705.

In step S701, the corresponding disease name entity is determined as the target disease in the medicine knowledge graph according to the keywords related to disease of the patient.

In an exemplary embodiment, if the disease keyword is “cold”, the disease name entity corresponding to “cold” is determined in the above medicine knowledge graph, and the cold can be regarded as the target disease.

In step S702, based on the medicine knowledge graph, all medicine name entities associated with the target disease are obtained as the first recommended medicines, and the medicine usage precaution entities associated with the first recommended medicines are also obtained.

Taking the above embodiment as an example, the description will be continued to obtain all medicine name entities associated with cold, and the medicines corresponding to these medicine name entities may be used as the first recommended medicines.

In step S703, the first recommended medicines are screened according to the keyword related to characteristics of the patient and the acquired medicine usage precaution entities, and second recommended medicines suitable for the patient are determined.

Taking the above embodiment as an example to continue the description, if the keyword related to characteristics of the patient includes “elderly people over 70 years old”, according to the medicine usage precaution entities of each medicine in the first recommended medicines, those medicines not suitable for the elderly are regarded as “unrecommended medicines”, and the remaining medicines after the screening can be regarded as the second recommended medicines for the patient.

In step S704, the second recommended medicines are screened according to the keyword related to the patient's response to medicines and the acquired medicine usage precaution entities to determine the target recommended medicine suitable for the patient.

Taking the above embodiment as an example to continue the description, if the keyword related to the patient's response to medicines includes “allergy to penicillin”, according to the medicine usage precaution entities of each medicine in the second recommended medicines, those medicines containing penicillin are regarded as “unrecommended medicines”, and the remaining medicines after the screening can be regarded as the target recommended medicines for the patients.

In step S705, based on the results of the above two screenings, the unrecommended medicines and the reasons for the non-recommendation are determined.

Taking the above embodiment as an example to continue the description, while obtaining the target recommended medicine after twice screenings, it is also possible to determine the unrecommended medicines and the reason for the non-recommendation. For example, when the second recommended medicines are obtained based on the first recommended medicines, the reason for filtering out the unrecommended medicine is “not suitable for the elderly”; when the target recommended medicine is obtained based on the second recommended medicines, the unrecommended medicines as filtered out include penicillin medicines and the reason for non-recommendation is “allergic to penicillin”. In this way, while outputting the target recommended medicines, the information of the unrecommended medicines determined here can also be output, thereby making it easier for the user to understand the specific situation and improving the user experience.

It should be noted that the execution order of steps S703 and S704 in the foregoing embodiment is not limited, and the order can be interchanged during actual execution. Furthermore, on the basis of steps S703 and S704, a screening step based on the above-mentioned keyword related to the patient's appeal can be further added. For example, medicines that patients do not want to use may be screened out, or medicines with the desired characteristics of patients are preferentially output during screening. Those skilled in the art can implement various combinations of screening steps on the basis of the above-mentioned embodiments, which will not be repeated here.

Continuing to refer to FIG. 2, after obtaining the target recommended medicine through the medicine filtering in the medicine knowledge graph, in step S203, the filtered-out target recommended medicine and unrecommended medicine information are output to complete the recommendation of the medicine.

In an exemplary embodiment, the name of the target recommended medicine is played in a voice manner, and/or the name of the target recommended medicine and the information of the unrecommended medicine are displayed in text.

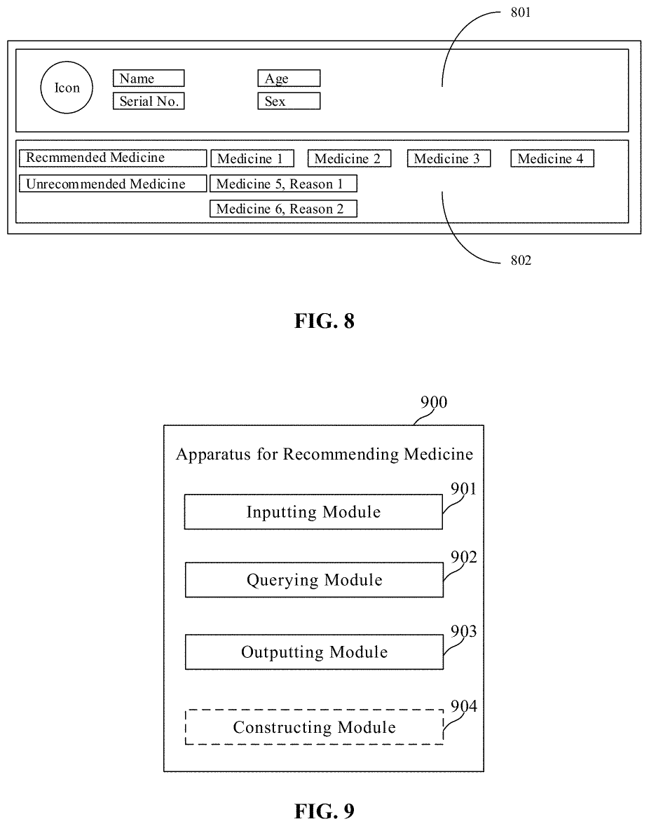

FIG. 8 schematically illustrates a medicine recommendation result output in text according to an exemplary embodiment of the present disclosure. Referring to FIG. 8, the information bar 801 is used for displaying the patient's icon (photo), name, serial number, age, sex, and other basic information, and the result bar 802 is used for displaying names of recommended medicines, names of unrecommended medicines, reasons for non-recommendation, and the like.

In the exemplary embodiment, according to the technical solution provided in this embodiment, various common diseases can be consulted, and recommended medicines for consulting diseases can be obtained, including the name of the medicine and the instructions for the medicine, as well as the doctor's guidance on the usage of medication (such as medicine interactions,) and other information. The medicine data in the medicine knowledge graph are interconnected by a network, so multiple medicines for the same disease can be extracted and displayed under a specific label, and common medicines for multiple diseases can also be extracted and displayed under another specific label. For a consultation keyword such as allergy history in the consultation information, if the keyword is retrieved in the medicine information of the medicine knowledge graph, the medicine may be hidden under the “unrecommended” label. According to the technical solution provided in this embodiment, the recommended medicine can be determined according to the user's consultation information for output, which helps the patient to understand the medicines, and can also help the doctor to get the medicine inspiration after obtaining the medicine recommendation list, thereby achieving high practical value.

The following describes apparatus embodiments of the present disclosure, which can be used to perform the above-mentioned method for recommending medicine of the present disclosure.

FIG. 9 schematically illustrates a block diagram of an apparatus for recommending medicine according to an exemplary embodiment of the present disclosure. Referring to FIG. 9, an apparatus 900 for recommending medicine provided in this embodiment includes an inputting module 901, a querying module 902, and an outputting module 903.

In an embodiment, the inputting module 901 is configured to acquire consultation information on medication of a patient, for example, received through keyboard input or voice input.

The above querying module 902 is configured to query a preset medicine knowledge graph based on the consultation information to filter out a target recommended medicine and unrecommended medicine information.

The outputting module 903 is configured to output the target recommended medicine and the unrecommended medicine information.

In an exemplary embodiment of the present disclosure, based on the foregoing solution, the unrecommended medicine information includes a name of a unrecommended medicine and a reason for non-recommendation.

In an exemplary embodiment of the present disclosure, based on the foregoing solution, the querying module 902 is specifically configured to: determine at least one consulting keyword based on the consulting information; input the at least one consulting keyword into the preset medicine knowledge graph, and perform medicine filtering through related information search in the medicine knowledge graph.

In an exemplary embodiment of the present disclosure, based on the foregoing solution, a mapping table between text and symbols is imported into the medicine knowledge graph, and the querying module 902 is further configured to: convert the consulting keyword into a symbol based on the mapping table; input the symbol into the medicine knowledge graph to perform direct match, and return a result of the medicine filtering.

In an exemplary embodiment of the present disclosure, based on the foregoing solution, the querying module 902 is specifically configured to obtain, from the consultation information on medication of the patient by means of natural language processing, at least one of following consultation keywords: a keyword related to the patient's disease, a keyword related to the patient's characteristics, a keyword related to the patient's appeal, or a keyword related to the patient's response to medicines.

In an exemplary embodiment of the present disclosure, based on the foregoing solution, the querying module 902 is specifically configured to determine, according to the keyword related to the patient's disease, a corresponding disease name entity as a target disease in the medicine knowledge graph; obtain, based on the medicine knowledge graph, all medicine name entities associated with the target disease as first recommended medicines, and obtain all medicine usage precaution entities associated with the first be recommended medicines; and determine the target recommended medicine applicable to the patient, a unrecommended medicine not applicable to the patient and a reason for non-recommendation by filtering the first recommended medicines according to at least one of the keyword related to the patient's characteristics, the keyword related to the patient's appeal, or the keyword related to the patient's response to medicines and the obtained medicine usage precaution entities.

In an exemplary embodiment of the present disclosure, based on the foregoing solution, the querying module 902 is specifically configured to: determine second recommended medicines applicable to the patient by performing a first filtering on the first recommended medicines according to the keyword related to the patient's characteristics and the medicine usage precaution entities; determine the target recommended medicine applicable to the patient by performing a second filtering on the second recommended medicines according to the keyword related to the patient's response to medicines and the medicine usage precaution entities; and determine the unrecommended medicine and the reason for non-recommendation based on results of the first filtering and the second filtering.

In an exemplary embodiment of the present disclosure, based on the foregoing solution, the keyword related to the patient's disease includes a name of a disease corresponding to the patient, the keyword related to the patient's characteristics includes age information and/or whether the patient is pregnant or not, the keyword related to the patient's appeal includes medicines that the patient does not want to use and/or characteristics of medicines required by the patient; and the keyword related to the patient's response to medicines includes allergy history information.

In an exemplary embodiment of the present disclosure, based on the foregoing solution, the outputting module 903 is configured to: output a name of the target recommended medicine by playing voice message, and/or, display the name of the target recommended medicine by text.

In an exemplary embodiment of the present disclosure, based on the foregoing solution, the above-mentioned apparatus further includes a constructing module 904 (shown by a dotted frame in the figure) configured to: collect medicine data, the medicine data including names of medicines, names of diseases to which the medicines apply, and precautions for usage of the medicines; and generate the preset medicine knowledge graph by determining a data triplet of “entity-relationship-entity” in which the names of diseases in the medicine data serve as a starting point, and the names of medicines corresponding to the starting point and the precautions for usage of the medicines are associated with the starting point.

In an exemplary embodiment of the present disclosure, based on the foregoing scheme, the entities in the data triplet include: a disease name entity, a medicine name entity, or a medicine usage precaution entity.

In an exemplary embodiment of the present disclosure, based on the foregoing scheme, the relationship in the data triplet includes: a treatment relationship between the disease name entity and the medicine name entity, or a usage description relationship between the medicine name entity and the medicine usage precaution entity.

In an exemplary embodiment of the present disclosure, based on the foregoing solution, the medicine data further includes a medicine dosage, and the outputting module 903 is further configured to output the medicine dosage along with the target recommended medicine to a user.

Since each functional module of the apparatus for establishing a treatment mode classification model according to the exemplary embodiment of the present disclosure corresponds to the steps of the exemplary embodiment of the method for establishing a treatment mode classification model described above, for details not disclosed in the apparatus embodiment of the present disclosure, reference can be made to the embodiment of the method for establishing the above treatment mode classification model of the present disclosure.

Since each functional module of the apparatus for recommending medicine according to exemplary embodiments of the present disclosure corresponds to the steps of the above exemplary embodiment of the method for recommending medicine, for details not disclosed in the apparatus embodiment of the present disclosure, reference can be made to the above exemplary embodiment of the method for recommending medicine.

Through the description of the above embodiments, those skilled in the art can easily understand that the example embodiments described here can be implemented by software, or can be implemented by software in combination with necessary hardware. Therefore, the technical solution according to the embodiments of the present disclosure may be embodied in the form of a software product, which may be stored in a non-volatile storage medium (e.g., CD-ROM, U disk, mobile hard disk, etc.) or on a network, including several instructions to cause a computing device (e.g., personal computer, server, mobile terminal, or network device, etc.) to perform the method according to the embodiments of the present disclosure.

Those skilled in the art can understand that various aspects of the present disclosure can be implemented as a system, a method, or a program product. Therefore, various aspects of the present disclosure may be specifically implemented in the form of a complete hardware implementation, a complete software implementation (including firmware, microcode, etc.), or a combination of hardware and software implementations, which may be collectively referred to herein as “circuit,” “module,” or “system.”

In an exemplary embodiment of the present disclosure, there is also provided a computer-readable storage medium on which a program product capable of implementing the above method of this specification is stored. In some possible implementations, various aspects of the present disclosure may also be implemented in the form of a program product, which includes program codes, and when the program product runs on a terminal device, the program codes are used to enable the terminal device to execute the steps according to various exemplary embodiments of the present disclosure described in the “Exemplary Method” section of the present specification.

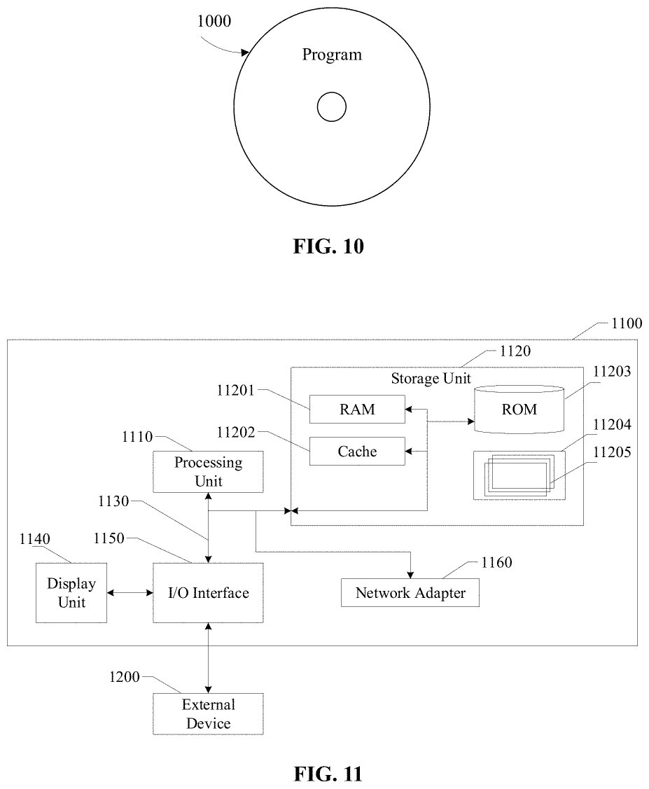

Referring to FIG. 10, a program product 1000 for implementing the above method according to an embodiment of the present disclosure is described, which may employ a portable compact disk read-only memory (CD-ROM) and include program codes, and may be included in a terminal device, for example, running on a personal computer. However, the program product of the present disclosure is not limited thereto, and in this document, the readable storage medium may be any tangible medium containing or storing a program, which may be used by or in combination with an instruction execution system, apparatus, or device.

The program product may employ any combination of one or more readable medium. The readable medium may be a readable signal medium or a readable storage medium. The readable storage medium may be, for example but not limited to, an electrical, magnetic, optical, electromagnetic, infrared, or semiconductor system, device, or any combination thereof. More specific examples of readable storage medium include (non-exhaustive list): electrical connections with one or more wires, portable disks, hard disks, random access memory (RAM), read only memory (ROM), erasable Programmable read-only memory (EPROM or flash memory), optical fiber, portable compact disk read-only memory (CD-ROM), optical storage device, magnetic storage device, or any suitable combination of the foregoing.

The computer-readable signal medium may include a data signal that is transmitted in baseband or as part of a carrier wave, in which readable program code is carried. This propagated data signal can take many forms, including but not limited to electromagnetic signals, optical signals, or any suitable combination of the foregoing. The readable signal medium may also be any readable medium other than a readable storage medium, and the readable medium may send, propagate, or transmit a program for use by or in combination with an instruction execution system, apparatus, or device.

The program code contained on the readable medium may be transmitted using any appropriate medium, including but not limited to wireless, wired, optical fiber cable, RF, etc., or any suitable combination of the foregoing.

Program codes for performing the operations of the present disclosure can be written in any combination of one or more programming languages including object-oriented programming languages such as Java, C++, etc., as well as conventional procedural programming language, such as “C” language or similar programming language. The program codes may be executed entirely on the user's computing device, partly on the user's device, as an independent software package, partly on the user's computing device and partly on a remote computing device, or entirely on the remote computing device or server for implementation. In situations involving remote computing devices, the remote computing device may be connected to the user computing device through any kind of network, including a local area network (LAN) or a wide area network (WAN), or may be connected to an external computing device (for example, using Internet service to connect via the Internet).

In an exemplary embodiment of the present disclosure, an electronic device capable of implementing the above method is also provided.

The electronic device 1100 according to this embodiment of the present disclosure is described below with reference to FIG. 11. The electronic device 1100 shown in FIG. 11 is only an example, and should not bring any limitation to the functions and use scope of the embodiments of the present disclosure.

As shown in FIG. 11, the electronic device 1100 is expressed in the form of a general-purpose computing device. Components of the electronic device 1100 may include, but are not limited to, at least one processing unit 1110, at least one storage unit 1120, and a bus 1130 that connects different system components (including the storage unit 1120 and the processing unit 1110).

In an embodiment, the storage unit stores a program code, and the program code can be executed by the processing unit 1110, so that the processing unit 1110 executes implementation steps according to various exemplary embodiment of the present disclosure as described in the “exemplary method” section of the specification. For example, the processing unit 1110 may execute, as shown in FIG. 1, step S101: acquire consultation information on medication of a patient; step S102: query a preset medicine knowledge graph based on the consultation information to filter out a target recommended medicine and unrecommended medicine information; and, step S103: output the target recommended medicine and the unrecommended medicine information.

The storage unit 1120 may include a readable medium in the form of a volatile storage unit, such as a random access storage unit (RAM) 11201 and/or a cache storage unit 11202, and may further include a read-only storage unit (ROM) 11203.

The storage unit 1120 may further include a program/utility tool 11204 having a set of (at least one) program modules 11205. Such program modules 11205 include but are not limited to: an operating system, one or more application programs, other program modules, and program data. Each of these examples or some combination may include an implementation of the network environment.

The bus 1130 may represent one or more of several types of bus structures, including a storage unit bus or a storage unit controller, a peripheral bus, a graphics acceleration port, a processing unit, or a local area using any of a variety of bus structures bus.

The electronic device 1100 may also communicate with one or more external devices 1200 (such as a keyboard, a pointing device, a Bluetooth device, etc.), and may also communicate with one or more devices that enable a user to interact with the electronic device 1100, and/or enable the electronic device 1100 to communicate with any other devices (e.g., a router, a modem, etc.) that communicates with one or more other computing devices. This communication can be performed through an input/output (I/O) interface 1150. Moreover, the electronic device 1100 may also communicate with one or more networks (such as a local area network (LAN), a wide area network (WAN), and/or a public network, e.g., the Internet) through the network adapter 1160. As shown, the network adapter 1160 communicates with other modules of the electronic device 1100 through the bus 1130. It should be understood that although not shown in the figure, other hardware and/or software modules may be used in conjunction with the electronic device 1100, including but not limited to: microcode, device driver, redundant processing unit, external disk drive array, RAID system, tape drive and data backup storage system.

Through the description of the above embodiments, those skilled in the art can easily understand that the example embodiments described here can be implemented by software, or can be implemented by software in combination with necessary hardware. Therefore, the technical solution according to the embodiments of the present disclosure may be embodied in the form of a software product, which may be stored in a non-volatile storage medium (e.g., CD-ROM, U disk, mobile hard disk, etc.) or on a network, including several instructions to cause a computing device (e.g., personal computer, server, terminal device, or network device, etc.) to perform the method according to the embodiments of the present disclosure.

In addition, the above-mentioned drawings are only schematic illustrations of the processing included in the method according to the exemplary embodiment of the present disclosure, and are not intended to limit the purpose. It is understood that the processes shown in the above drawings do not indicate or limit the chronological order of these processes. In addition, it is also easy to understand that these processes may be, for example, performed synchronously or asynchronously in multiple modules.

The technical solutions provided by the embodiments of the present disclosure may include the following beneficial effects.

In an embodiment of the present disclosure, the medicine knowledge graph is queried based on the acquired consultation information about the patient's medication to filter out and output the target recommended medicine and unrecommended medicine information, thereby improving the accuracy and efficiency of medicine recommendation and, thus, improving, user experience.

In another embodiment of the present disclosure, at least one consultation keyword is determined by obtaining the consultation information about the patient's medication, and input to the medicine knowledge graph to perform medicine filtering through related information search in the medicine knowledge graph. On one hand, medicine recommendation is implemented through the medicine knowledge graph constructed based on medical literature knowledge and clinical practical data to, thereby improving the accuracy of medicine recommendation. On the other hand, the associated query is performed in the medicine knowledge graph through at least one consultation keyword, the target recommended medicine corresponding to the above-mentioned consultation information can be efficiently obtained, thereby helping to improve the efficiency of medicine recommendation. On the other hand, while recommending applicable medicines, information about medicines that are not recommended is also output, including the name of the medicine and the reason for not recommending it, so as to make it easier for users to understand the specific situation and enhance the user experience.

After considering the description and practicing the invention disclosed herein, those skilled in the art will easily think of other embodiments of the present disclosure. This application is intended to cover any variations, uses, or adaptive changes of the present disclosure that follow the general principles of the present disclosure and include common general knowledge or customary technical means in the technical field not disclosed in the present disclosure. The description and examples are to be considered exemplary only, and the true scope and spirit of this disclosure are pointed out by the claims.

1. A method for recommending medicine performed by at least one computing device having at least one hardware processor and memory storing program instructions to be executed by the at least one hardware processor, the method comprising:
acquiring consultation information on medication of a patient;
querying a preset medicine knowledge graph based on the consultation information to filter out a target recommended medicine and unrecommended medicine information; and
outputting the target recommended medicine and the unrecommended medicine information.

2. The method for recommending medicine according to claim 1, wherein the unrecommended medicine information comprises a name of a unrecommended medicine and a reason for non-recommendation.

3. The method for recommending medicine according to claim 1, wherein querying the preset medicine knowledge graph based on the consultation information comprises:
determining at least one consulting keyword based on the consulting information;
inputting the at least one consulting keyword into the preset medicine knowledge graph; and
performing medicine filtering through a related information search in the medicine knowledge graph.

4. The method for recommending medicine according to claim 3, wherein a mapping table between text and symbols is imported into the medicine knowledge graph, and querying the preset medicine knowledge graph based on the consultation information further comprises:
converting the consulting keyword into a symbol based on the mapping table;
inputting the symbol into the medicine knowledge graph to perform direct match, matching; and
returning a result of the medicine filtering.

5. The method for recommending medicine according to claim 3, wherein determining at least one consultation keyword based on the consultation information comprises:
obtaining, from the consultation information on medication of the patient by natural language processing, at least one of following consultation keywords: a keyword related to the patient's disease, a keyword related to the patient's characteristics, a keyword related to the patient's appeal, or a keyword related to the patient's response to medicines.

6. The method for recommending medicines according to claim 5, wherein performing medicine filtering through the related information search in the medicine knowledge graph comprises:
determining, according to the keyword related to the patient's disease, a corresponding disease name entity as a target disease in the medicine knowledge graph;
obtaining, based on the medicine knowledge graph, all medicine name entities associated with the target disease as first recommended medicines, and obtaining all medicine usage precaution entities associated with the first recommended medicines; and
determining the target recommended medicine applicable to the patient, a unrecommended medicine not applicable to the patient and a reason for non-recommendation by filtering the first recommended medicines according to at least one of the keyword related to the patient's characteristics, the keyword related to the patient's appeal, or the keyword related to the patient's response to medicines and the obtained medicine usage precaution entities.

7. The method for recommending medicine according to claim 6, wherein filtering the first recommended medicines comprises:
determining second recommended medicines applicable to the patient by performing a first filtering on the first recommended medicines according to the keyword related to the patient's characteristics and the medicine usage precaution entities;
determining the target recommended medicine applicable to the patient by performing a second filtering on the second recommended medicines according to the keyword related to the patient's response to medicines and the medicine usage precaution entities; and
determining the unrecommended medicine and the reason for non-recommendation based on results of the first filtering and the second filtering.

8. The method for recommending medicine according to claim 5, wherein the keyword related to the patient's disease comprises a name of a disease corresponding to the patient, the keyword related to the patient's characteristics comprises at least one of: age information and whether the patient is pregnant or not, the keyword related to the patient's appeal comprises at least one of: medicines that the patient does not want to use and characteristics of medicines required by the patient; and the keyword related to the patient's response to medicines comprises allergy history information.

9. The method for recommending medicine according to claim 1, wherein outputting the target recommended medicine comprises at least one of: outputting a name of the target recommended medicine by playing voice message, and displaying the name of the target recommended medicine by text.

10. The method for recommending medicine according to claim 1, wherein, before querying a preset medicine knowledge graph based on the consultation information, the method further comprises:
collecting medicine data, the medicine data comprising names of medicines, names of diseases to which the medicines apply, and precautions for usage of the medicines; and
generating the preset medicine knowledge graph by determining a data triplet of “entity-relationship-entity” in which the names of diseases in the medicine data serve as a starting point, and the names of medicines corresponding to the starting point and the precautions for usage of the medicines are associated with the starting point.

11. The method for recommending medicine according to claim 10, wherein the entities in the data triplet comprise: a disease name entity, a medicine name entity, or a medicine usage precaution entity.

12. The method for recommending medicine according to claim 10, wherein the relationship in the data triplet comprises: a treatment relationship between the disease name entity and the medicine name entity, or a usage description relationship between the medicine name entity and the medicine usage precaution entity.

13. The method for recommending medicine according to claim 10, wherein the medicine data further comprises a medicine dosage, and the method further comprises: outputting the medicine dosage along with the target recommended medicine to a user.

14-18. (canceled)

19. An electronic device, including:
at least one hardware processor; and
a memory storing program instructions executable by the at least one hardware processor that, when executed, direct the at least one hardware processor to:
acquire consultation information on medication of a patient;
query a preset medicine knowledge graph based on the consultation information to filter out a target recommended medicine and unrecommended medicine information; and
output the target recommended medicine and the unrecommended medicine information.

20. (canceled)

21. The electronic device according to claim 19, wherein the unrecommended medicine information comprises a name of a unrecommended medicine and a reason for non-recommendation.

22. The electronic device according to claim 19, wherein the at least one hardware processor is further configured to:
determine at least one consulting keyword based on the consulting information;
input the at least one consulting keyword into the preset medicine knowledge graph; and
perform medicine filtering through a related information search in the medicine knowledge graph.

23. The electronic device according to claim 22, wherein the at least one hardware processor is further directed to:
import a mapping table between text and symbols into the medicine knowledge graph;
convert the consulting keyword into a symbol based on the mapping table;
input the symbol into the medicine knowledge graph to perform direct matching; and
return a result of the medicine filtering.

24. The electronic device according to claim 22, wherein the at least one hardware processor is further directed to:
obtain, from the consultation information on medication of the patient by natural language processing, at least one of following consultation keywords: a keyword related to the patient's disease, a keyword related to the patient's characteristics, a keyword related to the patient's appeal, and a keyword related to the patient's response to medicines.

25. The electronic device according to claim 19, wherein the at least one hardware processor is further directed to:
collect medicine data, the medicine data comprising names of medicines, names of diseases to which the medicines apply, and precautions for usage of the medicines; and
generate the preset medicine knowledge graph by determining a data triplet of “entity-relationship-entity” in which the names of diseases in the medicine data serve as a starting point, and the names of medicines corresponding to the starting point and the precautions for usage of the medicines are associated with the starting point.

26. A non-transitory computer-readable storage medium storing program instructions thereon that, when executed by at least one hardware processor, direct the at least one hardware processor to perform a method comprising:
acquiring consultation information on medication of a patient;
querying a preset medicine knowledge graph based on the consultation information to filter out a target recommended medicine and unrecommended medicine information; and
outputting the target recommended medicine and the unrecommended medicine information.
